(19) United States
(12) Patent Application Publication
(10) Pub. No.: US 2019/0192760 A1
(43) Pub. Date: Jun. 27, 2019

(54) SYSTEM AND DEVICE FOR INTELLIGENT BLADDER IRRIGATION AND METHOD FOR USING THE LIKE

(71) Applicant: Ilya Gorbachinsky, Las Vegas, NV (US)

(72) Inventor: Ilya Gorbachinsky, Las Vegas, NV (US)

(21) Appl. No.: 16/329,742

(22) PCT Filed: Dec. 22, 2017
(86) PCT No.: PCT/US17/61077

§ 371 Date: Feb. 28, 2019

Related U.S. Application Data
(60) Provisional application No. 62/420,963, filed on Nov. 11, 2016.

(51) Int. Cl. A61M 3/02; F04B 43/12

(52) U.S. Cl. CPC A61M 3/0283; A61M 3/022; A61M 25/0026; A61M 2205/3313; A61M 2210/1085; F04B 43/12

(57) ABSTRACT

A system and device for intelligent bladder irrigation, including an irrigation sensor structured to detect the presence of blood in an effluent liquid exiting a bladder during irrigation, a discrepancy mechanism structured to measure a difference between an inflow of an irrigant to a patient's bladder and an outflow of the effluent from the patient's bladder, and a flow varying mechanism for adjusting the inflow rate of the irrigant. The system also includes a control device structured to receive data from the irrigation sensor and the flow discrepancy mechanism, and generate a control signal in response.

### TECHNICAL FIELD

The present disclosure relates generally to bladder irrigation of patients in a hospital or other healthcare setting, more particularly to controlling irrigation rates during continuous bladder irrigation.

### BACKGROUND

In the urological field, blood clot formation in the bladder is of particular concern as the clots may cause blockages that can lead to bladder rupture or other negative outcomes, including death. As such, many conditions and procedures implicating a patient's bladder require irrigation in order to flush or wash the bladder during treatment or recovery. Specifically, continuous bladder irrigation (CBI) may be used to prevent clot formation in the bladder. CBI may also serve to enhance hemostasis and prevent occlusion of the indwelling catheter. Transurethral resection of the prostate (TURP) is one common type of urological procedure that typically requires continuous bladder irrigation treatment. For example, bladder tumors (of which, approximately 60,000 cases are diagnosed each year) almost always require TURP. Approximately another 90,000 additional procedures are performed each year (together, totaling approximately 150,000 procedures annually) to treat various other conditions as well. Even further, conditions that may cause intractable bleeding of the bladder, such as hemorrhagic cystitis, often necessitates CBI independent of surgical manipulation.

While medical technology has advanced rapidly in many areas, continuous bladder irrigation is still typically performed manually. Manual administration of this procedure requires healthcare providers to continuously monitor the patient's progress by inspecting the irrigation effluent at regular intervals, typically ranging from several minutes to several hours. Typically, the rate at which the irrigating fluid is delivered to the patient must be constantly adjusted in response to these observations. One known strategy involves turning a roller clap that regulates the flow of irrigation fluid (i.e., a Murphy drip).

Current procedures have several limitations and provide challenges for healthcare providers as the inflow rate can only be adjusted when the provider is in the room. These limitations can result in either excessive use of irrigating solution, which may require frequent changing of irrigation bags, or insufficient use, which may cause clot formation and possibly require surgical intervention. Further, current procedures are fairly inefficient as they tend to require continuous monitoring, cause inventory to be wasted, and compromise the health of both patients and healthcare providers.

One attempt to automate CBI is disclosed in PCT Publication No. WO 2017/112728 to Arun Rai et al. (“Rai”). Rai discloses an autonomous CBI strategy that includes a pressure sensor unit apparently configured to monitor changes in intra-abdominal pressure that may be indicative of a bladder occlusion in some instances. While this and other strategies for autonomous CBI may potentially be effective in detecting a bladder occlusion under optimal conditions, one of skill in the art will appreciate that pressure measurements by the pressure sensor unit described in Rai may be affected by external factors, such as a change in the patient's body position. As such, strategies for reliable, accurate autonomous CBI administration remain desirable.

### SUMMARY

In one aspect, a bladder irrigation system includes a catheter having an elongate tubular body positionable within a bladder of a patient, and having formed therein an inflow lumen for supplying an irrigant to the patient's bladder, and an outflow lumen for draining an effluent from the patient's bladder. The system also includes a sensor structured to sense a property of the effluent drained from the patient's bladder indicative of an irrigation status, a flow discrepancy mechanism structured to monitor a parameter indicative of a flow discrepancy between a flow of the irrigant to the patient's bladder and a flow of the effluent from the patient's bladder, a flow varying mechanism structured to vary a flow of the irrigant to the patient's bladder, and a control device structured to output a flow varying control signal responsive to at least one of the irrigation status or the parameter indicative of a flow discrepancy to vary a flow control state of the flow varying mechanism.

In another aspect, a device for controlling bladder irrigation in a patient includes a flow varying mechanism structured to vary a flow of an irrigant supplied to the patient's bladder, and a control device coupled with the flow varying mechanism, the control device being structured to determine an irrigation status from a parameter sensed by a sensor, detect an adverse bladder condition from a flow discrepancy observed by a flow discrepancy mechanism, and output a flow varying control signal responsive to at least one of the irrigation status or the adverse bladder condition to adjust a flow control state of the flow control mechanism.

In still another aspect, a method for bladder irrigation includes monitoring a first parameter indicative of at least one of a flow of an irrigant supplied to a patient's bladder or a flow of an effluent drained from the patient's bladder, monitoring a second parameter indicative of a property of the drained effluent, outputting a flow varying control signal responsive to at least one of the first parameter or the second parameter, and adjusting the flow of the irrigant supplied to the patient's bladder responsive to the flow varying control signal.

### BRIEF DESCRIPTION OF THE DRAWINGS

FIG. 1 is a diagrammatic view of a system for bladder irrigation, according to one embodiment;

FIG. 2 is a sectioned diagrammatic view of a catheter, according to one embodiment; and

FIG. 3 is a diagrammatic view of a control mechanism, according to one embodiment;

FIG. 4 is a diagrammatic view of a control mechanism, according to one embodiment;

FIG. 5 is a diagrammatic view of a control mechanism, according to one embodiment;

FIG. 6 is a flowchart illustrating example process and control logic, according to one embodiment;

FIG. 7 is a flowchart illustrating example process and control logic, according to one embodiment; and

FIG. 8 is a flowchart illustrating example process and control logic, according to one embodiment.

### DETAILED DESCRIPTION

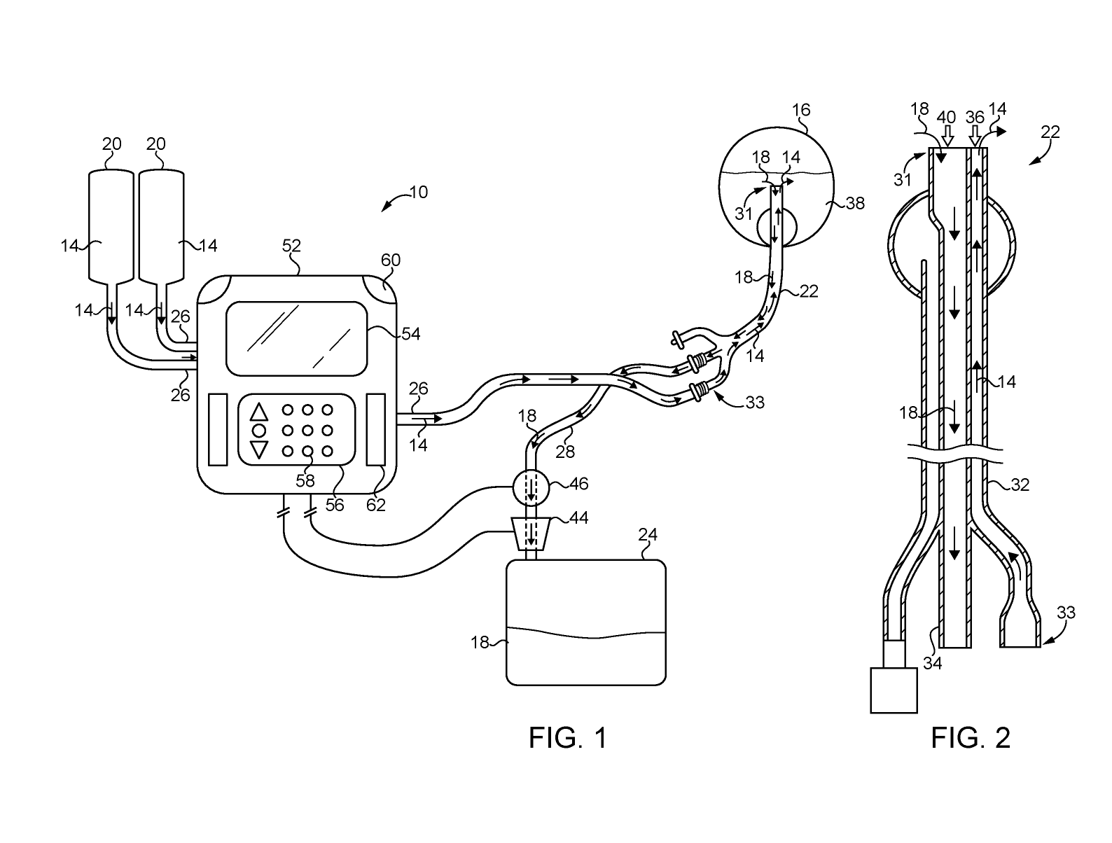

Referring to FIG. 1, an exemplary intelligent bladder irrigation system (“system”) 10 configured to facilitate a continuous bladder irrigation procedure (hereinafter, “procedure”) is shown according to one embodiment. It is contemplated that applications in which continuous bladder irrigation procedures are employed in the medical or hospital setting will particularly benefit from the teachings set forth herein; however, the present disclosure is not strictly limited to any particular setting or application. System 10 may include a fluid conduit 12 structured to supply a liquid irrigant solution (hereinafter, “irrigant”) 14 to a patient's bladder 16, and to drain a liquid effluent mixture (hereinafter, “effluent”) 18 therefrom.

Fluid conduit 12 may include an irrigant source 20 that contains an amount of irrigant 14, a catheter 22 having an elongate tubular body positionable within bladder 16, and an effluent container 24 for collecting effluent 18 drained from bladder 16. Irrigant source 20 is typically a gravity-fed intravenous (IV) drip bag preferably having a fluid capacity that ranges from about 3000 mL to about 5000 mL, but might be any other type of suitable irrigant container having a different fluid capacity. Irrigant 14 may include solutions consisting of sodium chloride 0.9%, aluminum ammonium sulphate, aluminum potassium sulphate, E -aminocaproic acid, or any other suitable irrigation solution. Fluid conduit 12 may further include one or more tubing lines, such as an inflow line 26 and an outflow line 28, that fluidly couple the individual components of fluid conduit 12. For example, inflow line 26 may fluidly couple irrigant source 20 with catheter 22 for supplying irrigant 14 to bladder 16, and outflow line 28 may fluidly couple catheter 22 with effluent container 24 for draining effluent 18 from bladder 16. The tubing lines each have formed therein, a lumen structured to allow liquids 14, 18 to flow therethrough, and may be formed of one or more clear and/or light -permeable materials suitable for use in the medical context, such as polyvinyl chloride (PVC), polyurethane, or another polymeric material. As seen in FIG. 1, irrigant source 20 may include a series of containers of irrigant 14, with each container being fluidly coupled to inflow line 26 by an adapter 30. In some embodiments, irrigant source 20 may include any other number of containers of irrigant 14, and/or may include an IV piggyback (IVPB) bag or other container to supply medicine, a different type of irrigant, or the like to bladder 16.

System 10 is configured such that irrigant 14 is supplied to bladder 16 from catheter 22 at a particular rate (hereinafter, “inflow rate”) that may be inputted, calculated, adjusted, or otherwise determined in a manner consistent with the discussion herein. System 10 may be gravity-assisted such that the relative position of each of the components of fluid conduit 12 may at least partially determine the inflow rate such that the maximum inflow rate may be achieved by allowing irrigant 14 to flow freely through inflow line 26 to catheter 22. In a normally proceeding procedure, the inflow rate may be expected to correspond with a rate at which effluent 18 is drained from bladder 16 through catheter 22 (hereinafter, “outflow rate”). In other embodiments, the flow of irrigant 14 into bladder 16 may be additionally and/or alternatively facilitated by a pump such as a peristaltic pump, a roller pump, or any other type of pump or other device suitable for use in a medical setting. It will be appreciated that the inflow rate and the outflow rate may be indicative of a flow or an amount of irrigant 14 supplied to bladder 16, and indicative of a flow or an amount of effluent 18 drained from bladder 16, respectively. As such, as used herein, “inflow rate” should be understood to include any measurement indicative of the amount of irrigant 14 supplied to bladder 16, such as a volume, mass, or weight, and “outflow rate” should be understood to include any measurement indicative of the amount of effluent 18 drained from bladder 16, such as a volume, mass, or weight.

Referring now also to FIG. 2, a cross section view of catheter 22 is shown. Catheter 22 may be a 3-way Y-shaped transurethral Foley catheter positionable within bladder 16, and may be formed of a flexible, fluid impermeable material suitable for use in the medical context, such as silicone, latex, PVC, polyurethane, or another polymeric material. Some embodiments of catheter 22 may also include a coating such as a silicon elastomer, hydrogel, or polytetrafluoroethylene (PTFE). In other embodiments, catheter 22 may be a 2-way Y-shaped catheter, a 4-way Y-shaped catheter, or any other type of catheter. Catheter 22 may have a distal end 31 that terminates in bladder 16 and a proximal end 33 structured for coupling catheter 22 with inflow line 26 and outflow line 28. Catheter 22 has formed therein, an inflow lumen 32 structured to receive irrigant 14 from inflow line 26, and an outflow lumen 34 structured to drain effluent 18 from bladder 16, each lumen 32, 34 extending between distal and proximal ends 31, 33. Inflow lumen 32 and outflow lumen 34 may terminate at an inflow opening 36 and an outflow opening 40, respectively, each opening 36, 40 being positioned at distal end 31. Inflow opening 36 fluidly couples inflow lumen 32 with bladder 16 so as to allow irrigant 14 to be discharged from catheter 22 into bladder 16, where irrigant 14 may mix with one or more bodily fluids 38, which includes urine (hereinafter, “urine 38”), to form effluent 18. Effluent 18 may evacuate bladder 16 through outflow opening 40, which fluidly couples bladder 16 to outflow lumen 34 so as to allow effluent 18 to be drained from bladder 16 to outflow line 28. Outflow line 28 may then carry effluent 18 to effluent container 24 for collection, monitoring, and/or disposal.

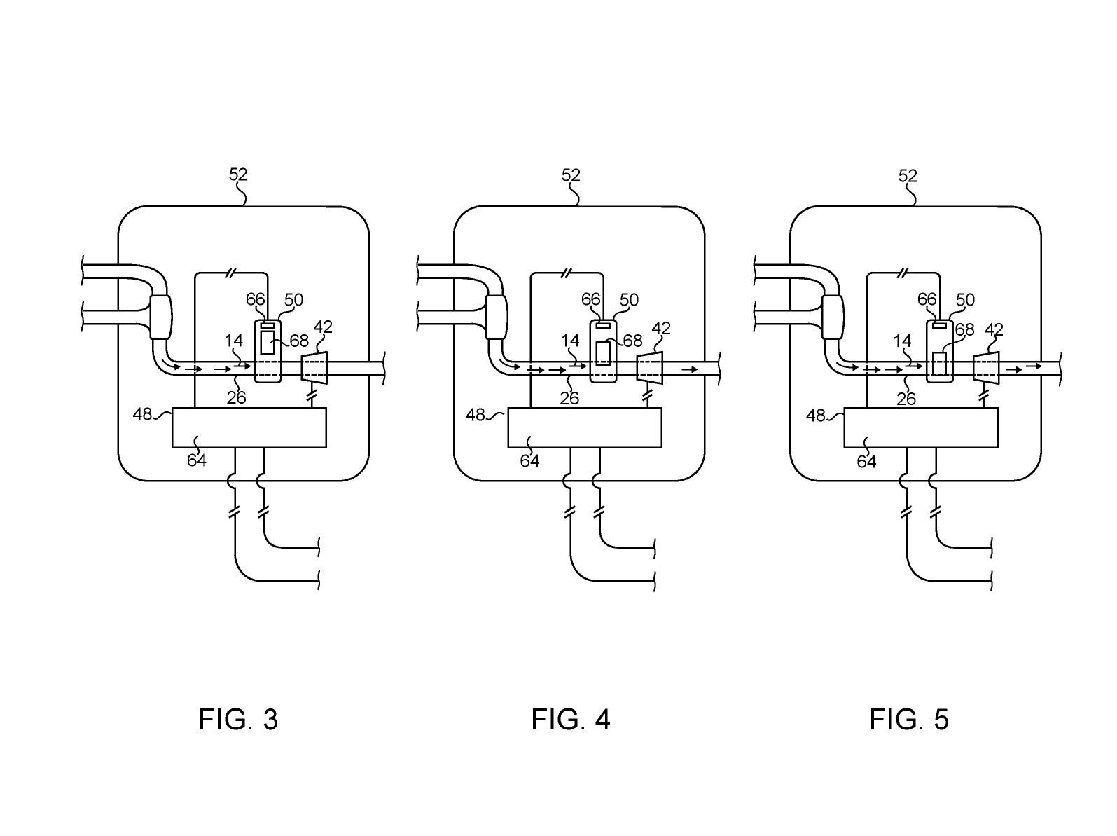

System 10 further includes one or more components that may be capable of acting in concert to sense or determine system parameters that are indicative of the progress or state of bladder irrigation (hereinafter, “irrigation status”), or system parameters that may be indicative of an adverse bladder condition, such as the presence of a bladder clot or other occlusion, a bladder rupture, or any other negative, unexpected, or otherwise undesirable outcome or condition implicated by the procedure, and adjusting the inflow rate in response. Components may include a flow discrepancy mechanism that has an inflow sensor 42 (illustrated in FIGS. 3-5, discussed hereinafter) coupled with inflow line 26 and an outflow sensor 44 coupled with outflow line 28, an irrigation sensor 46 coupled with outflow line 28, a control device 48 (illustrated in FIGS. 3-5, discussed hereinafter), and a flow varying mechanism 50 (illustrated in FIGS. 3-5, discussed hereinafter) coupled with inflow line 26.

In other embodiments, system 10 may include additional and/or alternative components for sensing one or more system parameters that may be indicative of an adverse bladder condition, such as a sensor structured to measure, detect, or monitor a system parameter indicative of system pressure, bladder pressure, effluent mass or volume, or fluid temperature, or for commanding, controlling, or otherwise varying delivery or conveyance of irrigant 14 to bladder 16, or draining effluent 18 from bladder 16. As seen in FIG. 1, system 10 may include an irrigation control module (ICM) 52 structured, at least in part, to package one or more system components together in a common housing, to allow for manual control of a procedure, or to display or input a system parameter such as, for example, sensor sensitivity, type of irrigant used, irrigant source capacity, effluent container volume, procedure duration, a target value such as a target effluent color or a target volume of irrigant to be supplied, or a predetermined threshold value such as maximum inflow rate or a maximum flow discrepancy. ICM 52 may include a display screen 54, a control panel 56 having one or more buttons 58 for inputting and/or controlling system parameters, and an alarm for outputting a warning notification or triggering other actions. The alarm may include lights 60 and speakers 62, and may be structured to warn a medical professional if, for instance, a predetermined threshold value may have been or is expected to be met, for example exceeded, or an occlusion or rupture may have or is expected to form in bladder 16. In other embodiments, the alarm may be structured to produce a different type of warning notification, such as a wireless signal, or any other form of visual, tactile, audio, or other type of notification. Display screen 54 may be a light-emitting diode (LED) display for displaying a user interface or for viewing system parameters or procedure information that may be of interest, such as, for example, an amount of irrigant 14 delivered to bladder 16, an amount of irrigant 14 remaining in irrigant source 20, an inflow rate, an amount of effluent 18 and/or urine 38 drained from bladder 16, or an outflow rate. In some embodiments, display screen 54 may be, for instance, a touch screen structured to allow a user to input one or more system parameters, or may be any other type of display screen suitable for use in the medical context. In still other embodiments, ICM 52 may include a printer for printing a physical copy of the system parameters and/or procedure information. Buttons 58 may be configured to allow a medical professional to input a system parameter, manually adjust the inflow rate, terminate the procedure, or input or adjust any other parameter or setting. In some embodiments, ICM 52 may include different and/or additional components for controlling or configuring the procedure, or may include additional ports, switches, or jacks.

Referring now also to FIGS. 3-5, diagrammatic views of an interior of ICM 52 are shown. ICM 52 may include control device 48, with each of inflow sensor 42, outflow sensor 44, and irrigation sensor 46 being coupled thereto in a manner that may allow each to send a signal encoding data indicative of a parameter of interest to control device 48, or be interrogated by control device 48 to produce the subject data. For instance, sensors 42, 44, 46 may be coupled with control device 48 by either a wired or a wireless connection. The flow discrepancy mechanism may be structured to monitor and compare the inflow rate and the outflow rate. Inflow sensor 42 may be configured to measure, detect, or monitor the inflow rate, which may be indicative of an amount of irrigant 14 supplied to bladder 16. For example, inflow sensor 42 may be a thermal mass-flow sensor having a heating element to heat a liquid and at least two temperature sensors to measure the temperature of the heated liquid downstream of the heating element, and may be structured to calculate a flow rate from the temperatures measured by the temperature sensors. In some embodiments, inflow sensor 42 may be structured to sense a different quantitative property indicative of the amount of irrigant 14 supplied to bladder 16, such as a mass or volume of supplied irrigant 14. Outflow sensor 44 may also be a thermal flow sensor and may be structured to measure, detect, or monitor the outflow rate, which may be indicative of an amount of effluent 18 drained from bladder 16. In some embodiments, outflow sensor 44 may be structured to sense a different quantitative property indicative of the amount of effluent 18 drained from bladder 16, such as a mass or volume of drained effluent 18. Outflow sensor 44 may be structured to measure, and/or control device 48 may be structured to calculate and/or factor in, a rate at which the patient is producing urine 38, or an amount of urine 38 the patient has produced during the procedure. In some embodiments, one or both of sensors 42, 44 may be a different type of flow meter, such as a variable area flow meter, a spring and piston flow meter, a mass gas flow meter, an ultrasonic flow meter, an optical flow meter, a pressure-based flow meter, a turbine flow meter, or may be any other type of suitable sensor. In other embodiments, one or both of sensors 42, 44 may be structured and/or positioned differently to measure, detect, or otherwise monitor a different quantitative property of irrigant 14 and effluent 18, respectively, such as temperature or weight. It will be appreciated, however, that some embodiments of the flow discrepancy mechanism may just monitor and compare the inflow and the outflow rates without ever calculating or otherwise considering the amounts or other quantities of irrigant 14 and/or effluent 18.

Irrigation sensor 46 may be positioned on or coupled with outflow line 28 and structured to sense a property of effluent 18 that may be indicative of the irrigation status of the procedure, wherein the irrigation status may indicate whether the inflow rate is sufficiently titrated to prevent an adverse bladder condition. In some embodiments, the property of effluent 18 sensed by irrigation sensor 46 may also be used by control device 48 to detect an adverse bladder condition. The sensed property of effluent 18 may include an optical property such as opacity or a color property. Irrigation sensor 46 may be a light-to -voltage converter, light-to-frequency converter, ambient light sensor, linear sensor array, color sensor, reflective light sensor, or any other type of sensor capable of identifying, detecting, or otherwise monitoring the property of interest. In an exemplary embodiment, irrigation sensor 46 includes a chromatic sensor structured to sense a concentration or an intensity of a reddish color indicative of an amount of blood in effluent 18. The sensitivity of sensors 42, 44, 46 may be configurable such that sensitivity can be tuned to the relevant system parameters. For example, irrigation sensor 46 may have a variety of sensitivity levels, with each suitable to detect or measure different colors, color intensities, color concentrations, or the like. In some embodiments, irrigation sensor 46 may be structured to sense a different color property, such as the presence of a predetermined color, the shade of a predetermined color, or the rate at which one or more colors permeate effluent 18. In other embodiments, irrigation sensor 46 may be structured to sense a different property of effluent 18. In still other embodiments, outflow sensor 44 may be packaged together with irrigation sensor 46 in a common housing, system 10 may instead include a single sensor coupled with outflow line 28 capable of monitoring both a property indicative of an amount of effluent 18 drained from bladder 16 and a property indicative of the irrigation status, or irrigation sensor 46 may be housed within outflow line 28 such that irrigation sensor 46 is replaced when outflow line 28 is replaced.

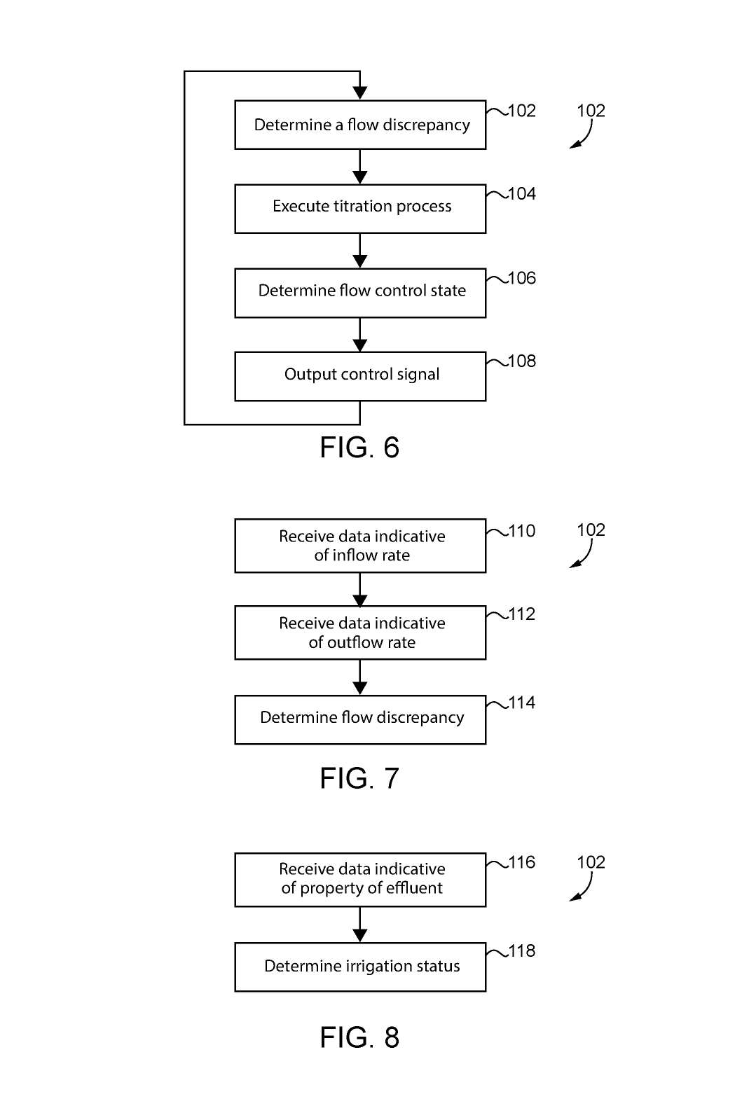

Control device 48 may be structured to output a flow varying control signal to command varying a flow control state of flow varying mechanism 50 based at least in part on data received by one or more of sensors 42, 44, 46. Control device 48 may include a memory (not pictured), a control logic 100 (as illustrated in FIGS. 6-8, discussed hereinafter), a user interface (not pictured) capable of being displayed on, for example, display screen 54, and a processor 64, or a series of processors, structured to perform calculations, execute instructions, communicate with other components of system 10 by, for example, sending or receiving signals, and/or perform other functions designed to facilitate control of system 10. Processor 64 might include a microprocessor or a field programmable gate array (FPGA), for example. The memory may be communicatively coupled with processor 64 and structured to store data and/or computer-executable instructions. The memory can include RAM, ROM, DRAM, SDRAM, Flash, or still other types of memory. Control device 48 may be a standalone unit structured for and dedicated to monitoring and/or adjusting the inflow rate, though, in other embodiments, control device 48 may be integrally formed with a shared control device structured to monitor or otherwise sense other health-related information of the patient, such as heart rate or blood pressure, or may be structured to perform other functions.

Flow varying mechanism 50 may be structured to adjust the inflow rate by selectively restricting a flow of irrigant 14 through inflow line 26. In some embodiments, flow varying mechanism 50 may additionally and/or alternatively be structured to at least partially adjust the inflow rate by other means. For example, flow varying mechanism 50 may include a pump or vacuum structured to facilitate and/or limit the inflow rate, or may be structured to adjust the height of an IV pole. It will be appreciated that in such embodiments, a flow varying control signal associated with a particular flow control state may be configured to adjust multiple system parameters associated with flow varying mechanism 50.

Flow varying mechanism 50 of FIGS. 3-5 includes a valve (hereinafter, “valve 50”). Valve 50 may include an electrical actuator 66 structured to actuate a pin 68 responsive to a flow varying control signal that may be outputted by control device 48. The flow varying control signal may energize electrical actuator 66 such that pin 68 is raised or lowered relative to inflow line 26, thereby blocking or unblocking, inflow line 26, respectively. In other embodiments, valve 50 may be hydraulically, pneumatically, or magnetically actuated, or may be a different type of valve, such as a cam operated valve, a stepper motor operated valve, a cartridge valve, a needle valve, or any other suitable type of valve structured to vary the inflow rate by any other means, including, for example, pinching, rolling, or the like. Valve 50 may include a plurality of flow control states, with each being associated with a different pin position relative to inflow line 26. The flow varying control signal may vary an energy state of electrical actuator 66 to change the flow control state of valve 50, which may correspond with a change in the inflow rate. For example, a third flow control state may be associated with a lower position of pin 68 than a first or a second flow control state, such that a flow varying control signal encoding a command to vary the flow control state to the third flow control state may lower the inflow rate by altering the orientation or position of valve 50 to restrict the flow of irrigant 14 through inflow line 26. It will be appreciated that, in like embodiments, a particular flow control state may correspond with similar or identical inflow rates across each embodiment. In other embodiments in which one or more components and/or system parameters may differ, like flow control states may correspond with different inflow rates in each embodiment. For example, in one embodiment, a flow control state associated with an inflow rate of 1.1 L/h using a first irrigant may correspond with an inflow rate of 1.2 L/h using a second irrigant where the second irrigant is slightly more viscous than the first irrigant. Similarly, in the present embodiment, a flow control state associated with an inflow rate of 1.1 L/h may be associated with an inflow rate of 1.2 L/h in an embodiment in which, for example, the inflow lumen has a larger diameter than that of inflow lumen 32.

As shown in FIG. 3, valve 50 can be seen in the first flow control state, which may be associated with an open configuration in which pin 68 may be raised such that irrigant 14 may be allowed to flow freely through inflow line 26, unobstructed by valve 50, which may correspond with a maximum inflow rate in certain embodiments. Referring now also to FIG. 4, valve 50 can be seen in the second flow control state, which may be associated with a partially lowered orientation in which pin 68 is lowered such that the flow of irrigant 14 through inflow line 26 may be partially obstructed but not stopped completely. Referring now also to FIG. 5, valve 50 is shown in the third flow control state, which may be associated with an orientation of valve 50 in which pin 68 may be fully lowered such that inflow line 26 is completely obstructed, thereby preventing or ceasing the flow of irrigant 14 through inflow line 26. Varying the flow control state may therefore vary the inflow rate in a manner consistent with the disclosure herein. It will be appreciated that in other embodiments, valve 50 may have a much greater number of flow control states, and that control device 48 may be structured to factor in one or more system parameters when outputting a flow varying control signal such that commanding the varying of the flow control state may adjust the inflow rate towards a target inflow rate.

Referring now also to FIG. 6, a flowchart illustrating process and control logic flow to determine a flow varying control signal for adjusting the flow control state is shown. Control device 48 may consider data from sensors 42, 44, 46 and may be configured by way of control logic 100 to adjust the flow control state in response. Control logic 100 may cause, when executed, control device 48 to perform a flow discrepancy process for detecting an adverse bladder condition, and a titration process for determining the irrigation status. Control device 48 may also be configured by way of control logic 100 to determine a flow discrepancy at block 102. In the flow discrepancy process, control device 48 may monitor data indicative of the inflow rate and data indicative of the outflow rate to detect the presence and/or measure the degree or magnitude of a discrepancy between the flow or amount of irrigant 14 supplied to bladder 16, and the flow or amount of effluent 18 drained from bladder 16 (hereinafter, “flow discrepancy”). As discussed above, irrigant 14 is typically continuously supplied to bladder 16 during a procedure and, in a normally proceeding procedure, the amount or flow of irrigant 14 supplied to bladder 16 should correspond with the amount or flow of effluent 18 drained from bladder 16. Control device 48 may also factor in and/or calculate a rate and/or volume of urine production to learn of the flow discrepancy. A flow discrepancy of a certain magnitude may indicate a problem with the procedure that might require assessment and/or action by a medical professional, such as an adverse bladder condition (e.g., a flow discrepancy may indicate a clot or bladder rupture that may result in irrigant 18 spilling into a body cavity), or it may indicate a problem with a system parameter such as, for instance, an empty irrigant source 20 or a full effluent container 24.

Referring now also to FIG. 7, a flowchart illustrating process and control logic flow for the flow discrepancy process is shown. Control device 48 may receive data indicative of the inflow rate from inflow sensor 42 at block 110, and may receive data indicative of the outflow rate from outflow sensor 44 at block 112. Control device 48 may then determine a flow discrepancy at block 114 by calculating a difference between the inflow rate and the outflow rate. Learning of the presence or magnitude of a flow discrepancy may allow control device 48 to detect an adverse bladder condition, which may include detecting the presence or absence of an adverse bladder condition, or may indicate an adverse bladder condition is likely or expected to occur. As mentioned previously, the inflow rate and the outflow rate may not be perfectly congruous in many instances as the outflow rate may be expected to exceed the inflow rate because urine 38 mixes with irrigant 14 before draining from bladder 16. As such, in some embodiments, control device 48 may be structured to calculate an expected flow discrepancy that factors in one or more system parameters, or to query an array of expected flow discrepancies programmed in the memory and select an expected flow discrepancy based on one or more system parameters. Control device 48 may be configured to compare the expected flow discrepancy to an observed or calculated flow discrepancy. In some embodiments, control device 48 may be structured to compare the observed or calculated discrepancy with a threshold flow discrepancy that has been inputted by a medical professional. Should control device 48 learn the flow discrepancy is above a threshold level or outside an expected flow discrepancy, or a range of expected flow discrepancies, control device 48 may log a default and determine an adjustment to the flow control state at block 106. Once the adjustment to the flow control state has been determined, control device 48 may output a flow varying control signal to adjust the flow control state at block 108. In determining an adjustment to the flow control state, the magnitude of any flow discrepancy may be considered, as well as the amount, intensity, or magnitude of any other parameters of interest. For example, a significant flow discrepancy (e.g., an observed flow discrepancy of 0.5 L/hr when the expected flow discrepancy was 0.1 L/hr) may be indicative of a bladder occlusion, which may result in control device 48 determining the flow control state should be adjusted to a flow control state similar or identical to the third flow control state illustrated in FIG. 5 in which valve 50 may be configured to cut off the flow of irrigant 14 to bladder 16. If, for example, control device 48 calculates or observes only a minor flow discrepancy (i.e., a flow discrepancy below a predetermined threshold, or a discrepancy within an expected range), control device 48 may determine no adjustment to the flow control state is necessary, or that a different adjustment should be made.

While control device 48 may be configured by way of control logic 100 to determine a flow discrepancy at block 102 and execute the titration process at block 104 in succession or even concurrently, control device 48 may be structured to at least temporarily prioritize the flow discrepancy process over the titration process or any other process that may be responsive to any other monitored parameter. Should control device 48 learn a flow discrepancy is above a threshold value or otherwise outside of an expected or acceptable range (i.e., a significant flow discrepancy), control device 48 may be structured to determine the flow control state at block 106 and output a control signal at block 108 based only on data resulting from the flow discrepancy process that may be indicative of a bladder occlusion or other adverse bladder condition, such as a flow discrepancy, which may need immediate attention by a medical professional, and which may be complicated further by additional amounts of irrigant 14 supplied to bladder 16. The alarm may be structured to produce an alarm notification responsive to at least one of the irrigation status or an adverse bladder condition. For example, should control device 48 detect an occlusion, the alarm may be configured to produce an alarm notification in response. In some embodiments, the alarm may be structured to produce an alarm notification responsive to an adjustment of the flow control state. In other embodiments, control device 48 may be structured to output an alarm control signal that in conjunction with or independent of a flow varying control signal. If control device 48 does not detect a flow discrepancy, detects a flow discrepancy is within an expected range, or detects a flow discrepancy below an inputted or predetermined threshold level, control device 48 may be configured by way of control logic 100 to execute the titration process at block 104. It will be appreciated, however, that the relative importance of the measurements or observations of the flow discrepancy and titration processes may depend on the extent to which the flow rates differ, the extent or nature of the color property of effluent 18, or any other property measured or observed. In some embodiments, data indicative of the inflow rate, the outflow rate, the color property of effluent 18, or other properties or parameters may be considered together or in any other manner that would prevent the same or other negative outcomes. For example, a bladder spasm may cause urine 38 to be pushed out of bladder 16 past catheter 22, which may cause disproportionate inflow and outflow rates but would not indicate a blockage or other complication. In other embodiments, control device 48 of the memory is programmed to execute the titration process and the flow discrepancy process in any other order.

Referring now also to FIG. 8, a flowchart illustrating process and control logic flow for executing the titration process 104 is shown. In the titration process, control device 48 may receive data indicative of a property of effluent 18 at block 116 to enable control device 48 to determine the irrigation status at block 118, which may indicate that irrigation of bladder 16 is proceeding as expected or if the inflow rate should be adjusted. In the exemplary disclosed embodiment discussed herein, the property of effluent indicative of an amount of blood in effluent 18 is a color property such as a concentration or intensity of a red color. The presence of a red color in effluent 18 may be caused by the presence of blood, which could result from a low inflow rate or excess bleeding. Put differently, the presence of a red color in effluent 18 of a certain concentration or intensity may indicate the bladder 16 is not being sufficiently irrigated, which may cause an amount of blood to drain from bladder 16 with effluent 18, and which may lead to clots that cause a bladder occlusion or other adverse bladder condition. Conversely, a low intensity or concentration of reddish color in effluent 18 may indicate an undesirably high inflow rate, which may potentially cause bladder 16 to become overfilled, and can lead to wasting irrigant 14, for instance. Control device 48 may also be configured by way of control logic 100 to determine an adjustment of the flow control state at block 106 by calculating or retrieving the target inflow rate and then adjusting the flow control state to a flow control state that corresponds therewith. The target inflow rate may be the inflow rate, or the range of inflow rates, suitable for titrating effluent 18 to produce a desired color property of effluent 18 consistent with a recommended therapy protocol. For example, effluent 18 having a rose color typically indicates irrigant 14 is being supplied to bladder 16 at a rate suitable to prevent both clotting and overfilling. The amount of any increase or decrease in inflow rate may depend on the concentration or intensity of the of the color property measured by irrigation sensor 46. Put differently, the inflow rate may be increased or decreased responsive to an intensity or a concentration of the red color sensed in effluent 18 such that the intensity or concentration of the red color increases or decreases to a desired level or range of levels. Control device 48 may be configured by way of control logic 100 to then output a flow varying control signal at block 108 that encodes for the determined adjustment to the flow control state. Some cycles of control logic 100 may not result in the varying of the flow control state and therefore no flow varying control signal may be generated if, for instance, the flow discrepancy process does not indicate a bladder occlusion and the titration process indicates the inflow rate is property titrated. Alternatively, a flow varying control signal could be produced but cause no change in the flow control state. Control device 48 may be programmed to run control logic 100 in a cyclical fashion at a predetermined interval, such as every 15-30 seconds, every minute, every hour, or any other desired interval. In some embodiments, the cycle period may be dependent on or otherwise factor in one or more system parameters, for example, if a bladder occlusion is suspected, an increased risk of bleeding or clot formation, or the patient's age or weight, control device 48 may be programmed to increase the frequency with which control logic 100 is run.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. It will be appreciated that certain features and/or properties of the present disclosure, such as relative dimensions or angles, may not be shown to scale. As noted above, the teachings set forth herein are applicable to a variety of different procedures and/or conditions having a variety of different medical applications than those specifically described herein. Other aspects, features and advantages will be apparent upon an examination of the attached drawings and appended claims. As used herein, the articles “a” and “an” are intended to include one or more items, and may be used interchangeably with “at least one.” Where only one item is intended, the term “one” or similar language is used. Also, as used herein, the terms “has,” “have,” “having,” or the like are intended to be open-ended terms.

What is claimed is:

1. A bladder irrigation system comprising:
a catheter having an elongate tubular body positionable within a bladder of a patient, and having formed therein an inflow lumen for supplying an irrigant to the patient's bladder, and an outflow lumen for draining an effluent from the patient's bladder;
a sensor structured to sense a property of the effluent drained from the patient's bladder indicative of an irrigation status;
a flow discrepancy mechanism structured to monitor a parameter indicative of a flow discrepancy between a flow of the irrigant to the patient's bladder and a flow of the effluent from the patient's bladder;
a flow varying mechanism structured to vary a flow of the irrigant to the patient's bladder; and
a control device structured to output a flow varying control signal responsive to at least one of the irrigation status or the parameter indicative of a flow discrepancy to vary a flow control state of the flow varying mechanism.

2. The system of claim 1 wherein the flow varying control signal varies the flow control state to change the flow of the irrigant to the patient's bladder.

3. The system of claim 1 wherein the flow varying mechanism includes an electrical actuator having an energy state that varies in response to the flow varying control signal.

4. The system of claim 1 wherein the property includes an optical property.

5. The system of claim 4 wherein the optical property includes a color property indicative of blood in the drained effluent, and the sensor further including a chromatic sensor structured to detect the color property.

6. The system of claim 5 wherein the color property includes at least one of a color intensity or a color concentration.

7. The system of claim 1 further including an alarm, wherein the alarm is structured to output an alarm notification responsive to at least one of the irrigation status or the parameter indicative of a flow discrepancy.

8. The system of claim 1 wherein the control device is structured to output a flow varying control signal to limit the flow of the irrigant to the patient's bladder if the flow discrepancy mechanism detects a flow discrepancy indicative of an occlusion or a bladder rupture.

9. The system of claim 8 wherein the control device is structured to output the flow varying control signal responsive only to the parameter indicative of a flow discrepancy and not responsive to the irrigation status.

10. A device for controlling bladder irrigation in a patient, the device comprising:
a flow varying mechanism structured to vary a flow of an irrigant supplied to the patient's bladder; and
a control device coupled with the flow varying mechanism, the control device being structured to:
determine an irrigation status from a parameter sensed by a sensor, and detect an adverse bladder condition from a flow discrepancy observed by a flow discrepancy mechanism; and
output a flow varying control signal responsive to at least one of the irrigation status or the adverse bladder condition to adjust a flow control state of the flow varying mechanism.

11. The device of claim 10 further including a sensor coupled with the control device and structured to detect an optical property of an effluent drained from the patient's bladder that is indicative of the irrigation status.

12. The device of claim 11 wherein the optical property includes a color property indicative of blood in the effluent drained from the patient's bladder.

13. The device of claim 10 further including a flow discrepancy mechanism structured to monitor a parameter indicative of a flow discrepancy between a flow of the irrigant to the patient's bladder and a flow of an effluent from the patient's bladder.

14. The device of claim 10 further including an alarm structured to output an alarm notification responsive to at least one of the irrigation status or the adverse bladder condition.

15. A method for bladder irrigation comprising:
monitoring a first parameter indicative of at least one of a flow of an irrigant supplied to a patient's bladder or a flow of an effluent drained from the patient's bladder;
monitoring a second parameter indicative of a property of the drained effluent;
outputting a flow varying control signal responsive to at least one of the first parameter or the second parameter; and
adjusting the flow of the irrigant supplied to the patient's bladder responsive to the flow varying control signal.

16. The method of claim 15 wherein the adjusting of the flow of the irrigant supplied to the patient's bladder occurs responsive to detection of a threshold level of the monitored first parameter or second parameter.

17. The method of claim 15 wherein the monitored second parameter is an optical property indicative of blood in the effluent.

18. The method of claim 17 wherein the optical property is a color intensity or color concentration.

19. The method of claim 15 further comprising triggering an alarm responsive to a flow discrepancy between the flow of the irrigant to the patient's bladder and the flow of the effluent from the patient's bladder that is indicative of an occlusion or a rupture of the patient's bladder.

20. The method of claim 15 wherein the adjusting of the flow of the irrigant to the patient's bladder further includes limiting the flow of the irrigant to the patient's bladder, and the flow varying control signal is responsive only to the first parameter and not the second parameter.
